(19) United States
(12) Patent Application Publication
(10) Pub. No.: US 2026/0224869 A1
(43) Pub. Date: Aug. 6, 2026

(54) METHOD AND KIT FOR TREATING DAMAGED CONNECTIVE TISSUE

(71) Applicant: Reddress Ltd., Pardes Hana (IL)

(72) Inventor: Alon KUSHNIR, Givat Ada (IL)

(73) Assignee: Reddress Ltd., Pardes Hana (IL)

(21) Appl. No.: 19/159,593

(22) PCT Filed: Aug. 31, 2023
(86) PCT No.: PCT/IL2023/050933

§ 371 Date: Aug. 25, 2025

(30) Foreign Application Priority Data
Mar. 2, 2023 (IL) 301095

(51) Int. Cl. A61M 35/00; A61M 1/00

(52) U.S. Cl. CPC A61M 35/00; A61M 1/772; A61M 2202/0413

(57) ABSTRACT

Growth or regeneration of a damaged connective tissue of a subject, achieved by combining the application of a support matrix and blood that is withdrawn from the subject and introduced in between the support matrix and the damaged portion of the connective tissue to form a coagulated blood mass there.

### TECHNOLOGICAL FIELD

The present disclosure is in the field of medical treatment of damaged connective tissues, particularly tendons, ligaments and cartilage.

### BACKGROUND ART

References considered to be relevant as background to the presently disclosed subject matter are listed below:
WO 2019/058373
WO 2019/058375
U.S. Pat. No. 9,180,142
WO 2001/32229
Markis EA, et al., Nature Reviews. Rheumatology, 23 Sep. 2014, 11(1):21-34

Acknowledgement of the above references herein is not to be inferred as meaning that these are in any way relevant to the patentability of the presently disclosed subject matter.

### GENERAL DESCRIPTION

The present disclosure provides, by one of its aspects, a method for treating or inducing growth or regeneration of a damaged connective tissue of a subject. by combining the application of a support matrix and blood that is withdrawn from the subject, introduced in between the support matrix and the damaged portion of the connective tissue to form a coagulated blood mass there. Provided by other aspects of this disclosure is a kit, support matrix and a conduit system, applicable in the method of this disclosure.

The term “said site” is used to denote the place of introduction of the whole blood between the support matrix and the damaged portion of the connective tissue.

The term “support matrix” is used herein to denote a matrix that is applied over a damaged portion of the connective tissue, which may be of a sheet, film, mesh, a wrap or a tube, made of collagen, a polymeric material that may be degradable, and in general any matrix that may be applied onto said damaged portion to assist in retaining the introduced whole blood at said site. The support matrix may, by some embodiments, be a matrix that is inert to the healing process, although, by other embodiments, the matrix may be made of a material that aids in the healing process. According to some embodiments of this disclosure the support matrix is positioned over the damaged connective tissue portion without fastening. By other embodiments, the support matrix is fastened to the tissue by stitching, by the use of surgical staples and/or by other means.

The blood is introduced in said site still in a flowable state, before fully coagulating, and the coagulated blood mass that is formed in said site and is in physical contact with the damaged portion of the connective tissue may improve the healing process, e.g., the regrowth of damaged connective tissue fibers, partial or full restoration of functionality, etc.

The connective tissue may, for example, be tendon, ligament or cartilage.

By one embodiment of the present disclosure, a support matrix is fitted over the damaged area of the connective tissue and blood that is withdrawn from the subject is introduced at said site, namely in between the support matrix and the damaged area using one or more catheters, tubes or other types of conduits.

The terms “conduit” is used herein to encompass catheters, tubes or other conduits. The term “conduit system” will be used to denote one or more conduits that are configured for joint application of whole blood to said site. The conduit system has a proximal end, connectable to an applicator for pushing blood into the conduit system and a distal end with one or more opening for delivering the blood to said site. The conduit system may comprise one set of such conduits used in the method disclosed herein. The conduit system typically comprises a main of feeder conduit connectable to the applicator and one or more application conduits. By some embodiments the conduit system comprises one application conduit, which may be a terminal segment of a single conduit that includes a proximal feeder segment, the terminal segment being often of a different, typically, narrower diameter than the feeder segment. By other embodiments the conduit system comprised a feeder conduit that branches into two or more downstream application conduits for delivering the blood to said site, each such application conduits having one or more opening at end section thereof through which the whole blood is applied to said site.

The term “applicator” is used herein to denote a device connectable to the feeder conduit, e.g., through a Luer-type connector.

During introduction, the conduit or conduit system may be gradually retracted to thereby fill the space between the support matrix and the damaged area with coagulating blood. Such introduction while retracting the conduit system distributes the introduced blood at said site, and may give rise to a substantially even distribution of the coagulating blood in said site, and particularly over the damaged area under said support matrix; and eventually a corresponding distribution of the coagulated blood mass. The introduction of the coagulating blood to said site and the subsequent retraction of the catheters are performed while the blood is flowable, namely before complete coagulation, namely before the coagulation process has been completed. The coagulation process is then completed while the blood is in said site.

The term “blood” is used herein (interchangeably with “whole blood”) to denote whole blood, typically venous blood withdrawn from the treated subject.

The term “coagulating blood” is used herein to denote whole blood that is still in a flowable state and is in the process of coagulation. A coagulating blood is typically whole blood mixed with one or more coagulating agents, one or more anti-anticoagulant (namely an agent that counteracts or inhibits the action of an anti-coagulant with which whole blood is mixed when withdrawn from a subject to avoid its coagulation) or a combination of one or more coagulating agents, one or more anti-anticoagulant.

The term “coagulated blood mass” is used herein to note the mass of coagulated blood formed form the introduced coagulating blood.

The term “coagulating agent” is used herein to denote one or more agents that induce coagulation or one or more anti-anticoagulants, namely agents that counteract the action of an anticoagulant that may be mixed with the withdrawn blood. Thus, the coagulating agent may comprise one or more agents that induce coagulation, one or more anti-anticoagulants or a combination thereof.

In an embodiment, the coagulating blood is a mixture of whole blood withdrawn from the subject with one or more coagulating agents and is introduced into the subject in between the support matrix and said site of the connective tissue, prior to the complete coagulation of the mixture, namely when the blood is still in a flowable state. The coagulation process of the mixture proceeds after injection and a coagulated blood mass is formed between the support matrix and the connective tissue.

In time, the coagulated blood mass may induce regeneration or growth of the damaged connective tissue.

Thus, in one aspect, the present disclosure provides a method for treating a damaged portion of a connective tissue, for example through inducing growth of connective tissue fibers across a damaged connective tissue portion. The method comprises: (a) fitting a support matrix onto said portion; (b) placing a distal end of one or more conduits between said portion and the support matrix; and (c) injecting a coagulating blood mass through a proximal end of the one or more conduits in between said portion and said support matrix, such that it accumulates and subsequently coagulates between the damaged portion of the connective tissue and the support matrix.

In one embodiment the one or more conduits are retraced during injection.

In one embodiment, said coagulating blood mass is a mixture of whole blood withdrawn from the subject and one or more coagulating agents and said introducing is performed prior to complete coagulation of the mixture, namely while it is still in a flowable form, and permitting the blood to coagulate on site.

It is to be noted, for any aspect of the present disclosure, that the whole blood may be withdrawn from the subject and initially mixed with anti-coagulating agents to prevent its natural coagulation. At a desired time, the whole blood is mixed with one or more coagulating agents that induces the coagulation process that will be completed at said site. The introduction of the coagulating blood into said site should be properly timed so the blood will still be in a flowable state, namely prior to complete coagulation.

In one embodiment, said connective tissue is a tendon a ligament or a cartilage. For example, the connective tissue can be the anterior cruciate ligament, or any tendon or a cartilage associated with the limbs e.g., the shoulder, knee, or the ankle.

In one embodiment, said placing is performed in parallel with fitting the support matrix onto the damaged region of the connective tissue.

In one embodiment, the one or more conduits are inserted in a minimally invasive procedure through a minimally invasive incision site.

In one embodiment, said coagulating blood mass is introduced into the conduits by an applicator, which may, for example, be a syringe.

In one embodiment, the conduit system for introducing the coagulating blood in the site between the support matrix and the damaged connective tissue portion comprise a feeding tube connectable to the applicator, e.g., by a Luer-type connector, that branches into two or more application tubes that are two or more conduits for introduction of the coagulating blood to said site.

In one embodiment, the support matrix is a patch or film; or is a tube or wrap capable of enveloping the damaged connective tissue.

In some embodiments of the method, the support matrix is selected from a group consisting of collagen, polyglycolic acid (PGA), polycaprolactone (PCL), polyvinyl alcohol (PVA), and porcine small intestine submucosa (SIS).

In one embodiment, the support matrix is a collagen sheet, film, or wrap.

In one embodiment, the damaged connective tissue portion comprises cut connective tissue fibers.

In one embodiment, said connective tissue portion includes two cut connective tissue stumps that are surgically joined or brought into proximity.

In one embodiment, said joining or bringing into proximity, comprises suturing the two stumps to said support matrix.

In one embodiment, said introducing is carried out such that said coagulating blood mass comes into tight contact with said stumps.

In some embodiments, the method of the invention further comprises passing oxygen-containing gas through the blood (e.g., to saturate the blood with oxygen), prior to introducing it into said site. The contact of the blood with the oxygen-containing gas may be prior to or after the addition of a coagulating agent. The oxygen-containing gas may be air or an oxygen-rich gas, e.g., oxygen-enriched air or pure oxygen.

In some embodiments, additional components may be added to the blood. Non-limiting examples include keratin, growth factors, bone marrow, gases, or cells, e.g., stem cells.

In some embodiments, the method of the invention further comprises mixing the blood, prior to said introducing, with one or more autologous cells.

In another aspect, the present invention provides a kit for inducing growth or regenerating a damaged connective tissue portion of a subject, the kit comprising: (i) one or more blood withdrawal devices for allowing withdrawal of blood from the subject; (ii) one or more blood collection receptacles for receiving the blood withdrawn from the subject; and (iii) a coagulation assembly configured for permitting mixture of the withdrawn blood with one or more coagulation agents for initiating coagulation process of the withdrawn blood; and a conduit system of the kind described above that is configured for delivering the coagulating blood into said site. The kit may optionally comprise (a) an applicator for introducing the coagulating blood into said conduits; and (b) instructions for use of the kit for inducing growth or regenerating a damaged connective tissue portion of a subject.

In one embodiment, said kit further comprises a support matrix.

In one embodiment the one or more collection receptacles are vacuum tubes.

In one embodiment, the coagulation assembly comprises a device for contacting the withdrawn blood with one or more coagulating agents.

In one embodiment, the kit further comprises one or more anticoagulants for mixing with the withdrawn blood to avoid its uncontrolled coagulation.

In one embodiment the one or more anticoagulants are comprised within said one or more blood collection receptacles.

In one embodiment, the anticoagulant selected from a group consisting of: ACD-A (Anticoagulant Citrate Dextrose, Solution A), EDTA (ethylenediaminetetraacetic Acid), EGTA (ethylene glycol tetra acetic acid), a citrate, Heparin, and oxalate.

In one embodiment, the one or more coagulation agents or anti-anticoagulants are selected from: Kaolin, Ca²⁺, e.g., in the form of calcium salts such as Calcium Gluconate, Mg²⁺, negatively charged phospholipid (PL) and protamine sulfate.

In one embodiment the one or conduits are connectable at their proximal end to an applicator.

In one embodiment the kit comprises a conduit system for introducing the coagulating blood in the site between the support matrix and the damaged connective tissue portion that comprises a feeding tube connectable to the applicator, e.g., by a Luer-type connector, that extends into two or more application tubes that are two or more conduits for introduction of the coagulating blood to said site.

In one embodiment, said damaged connective tissue portion for which the kit is used is a cut connective tissue having a first and second connective tissue stumps.

In one embodiment, the kit further comprises sutures, surgical staplers or other means for securing said damaged connective tissue portion to the support matrix.

Said connective tissue for which the kit is used may be a ligament, tendon or cartilage.

In another aspect, the present disclosure provides a kit for treating a damaged tissue portion (e.g., inducing growth or regenerating a damaged connective tissue portion) of a subject, the kit comprising (in any applicable order): (i) fitting a support matrix onto a damaged region of the connective tissue; (ii) placing a distal end of one or more conduits between the damaged region of the connective tissue and the support matrix; (iii) withdrawing whole blood from the subject; (iv) mixing the subject's blood with one or more coagulation agents or anti-anticoagulation agents; (v) prior to complete coagulation of the blood, injecting the blood with the coagulation agent through a proximal end of the one or more conduits; and (vi) permitting the blood to coagulate between the damaged connective tissue and the support matrix.

In one embodiment, steps (iii) and (iv) are performed prior to or concomitantly with steps (i) and (ii).

By some embodiments, the kit is usable in a method as described above.

Also provided by this disclosure is a support matrix for use in treating a damaged connective tissue or inducing growth of connective tissue fibers across a damaged connective tissue portion in a subject in need thereof, the treatment comprising associating said matrix with a damaged portion of the connective tissue, and introducing coagulating blood in between said matrix and said damaged portion.

The support matrix of this disclosure may be used in a method that comprises: (a) fitting the support matrix onto said portion; (b) placing a distal end of one or more conduits between said portion and the support matrix; and (c) injecting a coagulating blood mass through a proximal end of the one or more conduits in between said portion and said support matrix (such that it accumulates and subsequently coagulates between the damaged portion of the connective tissue and the support matrix).

The support matrix may be in the form of a patch or film. It may also be in the form of a tube or a wrap capable of enveloping the damaged connective tissue portion. The support matrix may be made of a collagen sheet, film, or wrap.

The support matrix of this disclosure may be used in a method as described above.

Also provides by this disclosure is a conduit system for use in treating a damaged connective tissue or inducing growth of connective tissue fibers across a damaged connective tissue portion in a subject in need thereof, the treatment comprising associating said matrix with a damaged portion of the connective tissue, and introducing coagulating blood at site in between said matrix and said damaged portion; the conduit system connectable to an applicator at its proximal end and having one or more openings at a distal end for introducing the of coagulating blood into said site.

By one embodiment, the conduit system comprises a feeding conduit that branches, typically through a multifurcation element, into two or more downstream application conduits for delivering the blood to said site.

By one embodiment, the conduit systems is provided in a kit of parts including also an applicator.

### EMBODIMENTS

Some embodiments of this disclosure will now be described in the following numbered paragraphs and referred to in subsequent paragraphs by reference to the numbers (in a format similar to that of claims). These embodiments intend to add onto (and not limit) the disclosure herein:
1. A method for treating a damaged connective tissue portion (e.g., for inducing growth of connective tissue fibers across a damaged portion of connective tissue) in a subject, comprising:
   (a) fitting a support matrix onto said portion;
   (b) placing a distal end of one or more conduits between said portion and the support matrix; and
   (c) injecting a coagulating blood mass through a proximal end of the one or more conduits in between said portion and said support matrix, such that it accumulates and subsequently coagulates between the damaged portion of the connective tissue and the support matrix.
2. The method of embodiment 1, wherein the one or more conduits are retraced during injection.
3. The method of embodiment 1 or 2, wherein said coagulating blood mass is a mixture of whole blood withdrawn from the subject and one or more coagulating agents, and wherein said introducing is performed prior to complete coagulation of the mixture.
4. The method of any one of embodiments 1 to 3, wherein said connective tissue is a tendon a ligament or a cartilage.
5. The method of any one of embodiments 1 to 4, wherein said placing is performed in parallel with fitting the support matrix onto the damaged region of the connective tissue.
6. The method of any one of embodiments 1 to 5, wherein said conduit is a tube or a catheter.
7. The method of embodiment 6, wherein said one or more conduits are inserted and positioned through a minimal incision site.
8. The method of any one of embodiments 1 to 7, wherein said coagulating blood mass is introduced into the conduits by an applicator.
9. The method of embodiment 8, wherein said applicator is a syringe.
10. The method of any one of embodiments 1 to 9, wherein said support matrix is a patch mesh, sheet, membrane, film, for placing onto a damaged portion of the connective tissue, e.g., covering it.
11. The method of any one of embodiments 1 to 9, wherein said support matrix is a tube or wrap for placing around a damaged portion of a connective tissue, e.g., around a damaged tendon or ligament.
12. The method of any one of embodiments 1 to 11, wherein said support matrix is made of collagen.
13. The method of any one of embodiments 1 to 12, wherein the damaged connective tissue portion comprises cut connective tissue fibers.
14. The method of embodiment 13, wherein said damaged connective tissue portion includes two cut connective tissue stumps that are surgically joined or brought into proximity to one another.
15. The method of embodiment 14, wherein said joining or bringing into proximity, comprises suturing the two stumps to said support matrix.
16. The method of embodiment 14 or 15, wherein said introducing is carried out such that said coagulating blood mass comes into contact with said stumps.
17. The method of any one of embodiments 1 to 16, wherein said method further comprises passing oxygen-containing gas through the blood.
18. The method of any one of embodiments 1 to 17, further comprising mixing the blood, prior to said introducing, with one or more autologous cells.
19. A kit for use in treating a damaged tissue portion (e.g., inducing growth or regenerating a damaged connective tissue portion) of a subject, the kit comprising:
   one or more blood withdrawal devices for withdrawing blood from the subject;
   one or more blood collection receptacles for receiving the blood withdrawn from the subject;
   a device for mixing of the withdrawn blood with one or more coagulation agents to initiate coagulation of the withdrawn blood;
   one or more conduits (e.g., tubes or catheters) for delivering coagulating blood in between a support matrix and said portion; optionally one or both of
   one or more applicators couplable to the one or more conduits for introducing the coagulating blood into said conduits; and optionally
   instructions for use of the kit for inducing growth or regenerating a damaged connective tissue portion of a subject.
20. The kit of embodiment 19, wherein said kit further comprises a support matrix.
21. The kit of embodiment 20, wherein the support matrix is a patch mesh, sheet, membrane, film, tube or wrap.
22. The kit of embodiment 21, wherein the support matrix comprises or is made entirely of collagen.
23. The kit of any one of embodiments 19 to 22, comprising a conduit system connectable/couplable to an applicator at its proximal end and having one or more openings at a distal end for introducing the coagulating blood into said site.
24. The kit of embodiment 23, wherein the conduit system comprises a feeding conduit that branches into two or more downstream application conduits for delivering the blood to said site.
25. The kit of any one of embodiments 21 to 24, comprising an applicator couplable to said conduit system for feeding blood thereinto.
26. The kit of any one of embodiments 19 to 25, wherein said one or more blood collection receptacles are vacuum tubes.
27. The kit of any one of embodiments 19 to 26, wherein the coagulation assembly comprises a device for contacting the withdrawn blood with one or more coagulating agents.
28. The kit of any one of embodiments 19 to 27, further comprising one or more anticoagulation agents for mixing with the withdrawn blood.
29. The kit of embodiment 28, wherein the one or more anticoagulants are comprised within said one or more blood collection receptacles.
30. The kit of embodiment 28 or 29, wherein the anticoagulant selected from a group consisting of: ACD-A (Anticoagulant Citrate Dextrose, Solution A), EDTA (ethylenediaminetetraacetic Acid), EGTA (ethylene glycol tetra acetic acid), a citrate, Heparin, and oxalate.
31. The kit of any one of embodiments 19 to 30, wherein said one or more coagulation agents or anti-anticoagulating agents are selected from: Kaolin, Ca2+, Mg2+, negatively charged phospholipid (PL) and protamine sulfate.
32. The kit of any one of embodiments 19 to 31, wherein said damaged connective tissue portion is a cut connective tissue having a first and second connective tissue stumps.
33. The kit of any one of embodiments 19 to 32, further comprising securing elements for securing said damaged connective tissue portion to the support matrix.
34. The kit of any one of embodiments 19 to 33, wherein said damaged connective tissue portion is a cut connective tissue having a first and second connective tissue stumps.
35. The kit of any one of embodiments 19 to 34, wherein said connective tissue is a ligament, tendon or cartilage.
36. The kit of any one of embodiments 19 to 35, for use in a method for treating, inducing growth, or regenerating a damaged connective tissue portion, the method comprising:
   (i) fitting a support matrix onto a damaged region of the connective tissue;
   (ii) placing a distal end of one or more conduits between the damaged region of the connective tissue and the support matrix,
   (iii) withdrawing whole blood from the subject;
   (iv) mixing the subject's blood with one or more coagulation agents;
   (v) prior to complete coagulation of the blood, injecting the blood with the coagulation agent through a proximal end of the one or more conduits; and
   (vi) permitting the blood to coagulate between the damaged connective tissue and the support matrix.
37. The kit of embodiment 35 wherein steps (iii) and (iv) are performed prior to or concomitantly with step (i) and (ii).
38. The kit of any one of embodiments 19 to 37, for use in a method according to any one of claims 1 to 18.
39. A support matrix for use in treating a damaged connective tissue or inducing growth of connective tissue fibers across a damaged connective tissue portion in a subject in need thereof, the treatment comprising associating said matrix with a damaged portion of the connective tissue, and introducing coagulating blood in between said matrix and said damaged portion.
40. The support matrix of embodiment 39, for use in a method that comprises:
   (a) fitting the support matrix onto said portion;
   (b) placing a distal end of one or more conduits between said portion and the support matrix; and
   (c) injecting a coagulating blood mass through a proximal end of the one or more conduits in between said portion and said support matrix (such that it accumulates and subsequently coagulates between the damaged portion of the connective tissue and the support matrix).
41. The support matrix of embodiment 40, wherein the one or more conduits are retraced during injection.
42. The support matrix of embodiment 40 or 41, wherein said coagulating blood mass is a mixture of whole blood withdrawn from the subject and one or more coagulating agents, and wherein said introducing is performed prior to complete coagulation of the mixture.
43. The support matrix of any one of embodiments 40 to 42, in the form of a patch or film.
44. The support matrix of any one of embodiments 40 to 42, in the form of a tube or a wrap capable of enveloping the damaged connective tissue portion.
45. The support matrix of any one of embodiments 40 to 44, being a collagen sheet, film, or wrap.
46. The support matrix of any one of embodiments 40 to 45 for use in a method of any one of embodiments 1 to 18.
47. A conduit system for use in treating a damaged connective tissue or inducing growth of connective tissue fibers across a damaged connective tissue portion in a subject in need thereof, the treatment comprising associating said matrix with a damaged portion of the connective tissue, and introducing coagulating blood at site in between said matrix and said damaged portion; the conduit system connectable to an applicator at its proximal end and having one or more openings at a distal end for introducing the of coagulating blood into said site.
48. The conduit system of embodiment 47, comprising a feeding conduit that branches into two or more downstream application conduits for delivering the blood to said site.
49. The conduit system of embodiment 47 or 48, provided in a kit of parts comprising also an applicator couplable to said conduit system for feeding blood to said system.
50. The conduit system of any one of embodiments 47 to 49, for use in a method of any one of embodiments 1 to 18.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIG. 1 is a schematic illustration of an exemplary embodiment of this disclosure, following arthroscopic placement of a regeneration-supporting matrix onto a shoulder tendon. The illustration shows a tube which was inserted through one of the openings that were formed for the arthroscopic procedure and through which coagulating blood is to be introduced, and which is still in position.

FIG. 2 is a schematic illustration, showing the shoulder tendon (with the skin removed for illustrative purposes), with the tube's distal end in position between the matrix and the tendon, according to an embodiment of this disclosure.

FIG. 3 is a schematic illustration of several (three in this example) distal tubes connecting to a single multifurcation element.

FIGS. 4A and 4B are schematic illustrations of an exemplary embodiment of this disclosure, for the treatment of damaged cartilage.

FIG. 5 is a block diagram of procedure according to an exemplary embodiment of this disclosure.

### DESCRIPTION OF SPECIFIC EMBODIMENTS

Thus, an aspect of the present disclosure provides a method for regenerating a connective tissue in or across a damaged connective tissue portion. The method includes fitting a support matrix over the damaged area. The support matrix may be in the form of a patch or in the form of a hollowed element having a lumen capable of wrapping around or enveloping the damaged tissue portion, e.g., a collagen sheet or wrap. The support matrix is intended to cover or envelope the damaged connective tissue portion and the procedure being designed to allow introduction of coagulating blood withdrawn from the subject to the enclosed space to form a blood clot on the damaged connective tissue.

To allow uniform spread of the coagulating blood within the space, the distal end of one or more tubes or catheters are placed between the damaged region of the connective tissue and the support matrix. The proximal end of the tubes or catheters protrudes outside the subject's body.

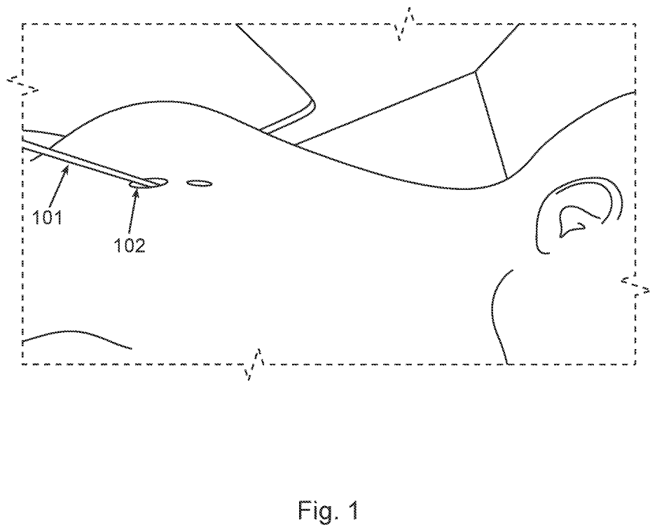

Reference is being made to FIG. 1, which is a schematic illustration showing a portion of a catheter 101 that protrudes (i.e., extends out of) the incision site 102 and which is temporarily retained within the subject after the implant procedure and before the injection of the coagulating blood.

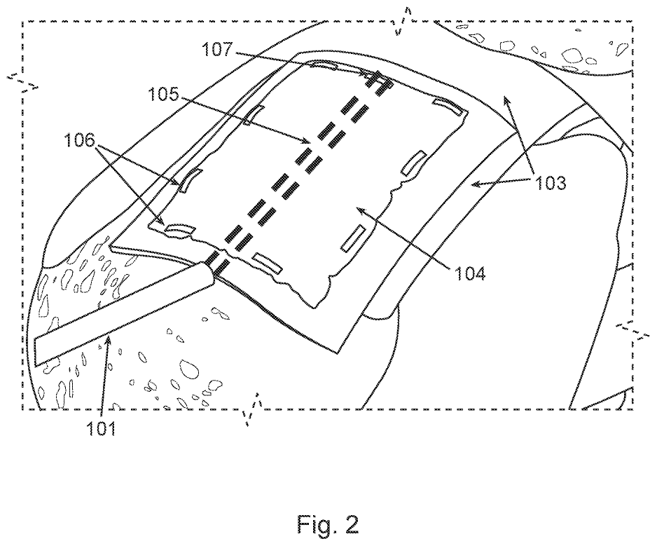

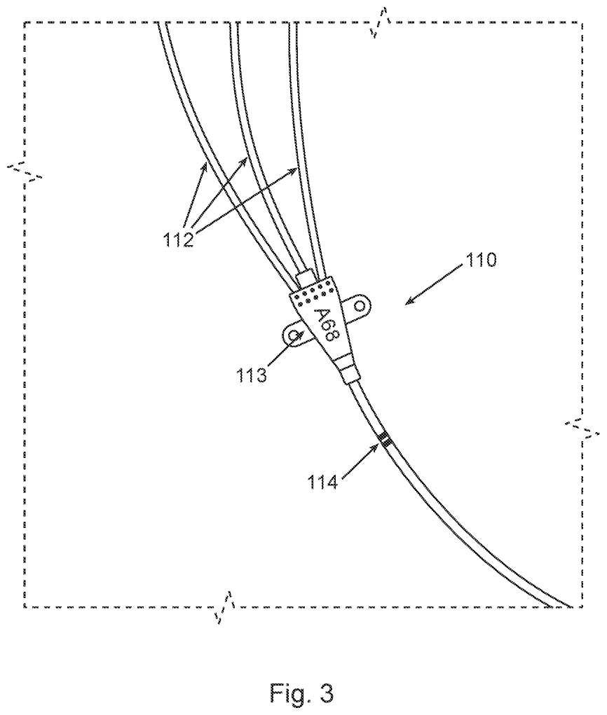

Reference is being made to FIG. 2, which is a schematic illustration showing a portion of a distal segment of a catheter 101 within the subject's body, the distal end portion thereof, represented by dashed lines 105 being positioned between the regeneration-supporting matrix 104, for example a collagen-based matrix, fixed using surgical anchoring elements 106 and the tendon 103. In this illustrative embodiment, catheter 101 is positioned such that its distal end 107 is close to the distal end of support matrix 104. By some embodiments a tube or catheter with a single distal segment 105 is used. In other embodiments a catheter with multiple distal segments may be used, such as the catheter 110, shown in FIG. 3, having a single proximal segment 111 (referred to above as “feeder conduit”) and multiple distal segments 112 (3 in this embodiment, referred to above as “application conduits”) extending from a multifurcation element 113. The one or more distal segments may have a single opening at their end; or in other embodiments the end portion of the distal segment may have each two or more openings, e.g., one end opening and one or more formed in the wall of the end portion, to permit egress of the coagulating blood in more than one location along the end portion. Where the distal segment has one opening at the distal end, the distal end may be placed below a mid-portion of the matrix 104, whereby the injected coagulating blood will be spread during injection to surrounding portions below the matrix; or alternatively, the distal segment may be gradually withdrawn during injection to thereby spread the coagulating blood below the matrix. It is also possible by other embodiments to use multiple catheters with their distal ends being distributed in different locations between the connective tissue and the matrix to assure adequate spread of a coagulating blood mass.

An applicator comprising the coagulating blood mass, e.g., a syringe, is then connected to the protruding proximal end of the tubes or catheters, and the coagulating blood mass may then be injected.

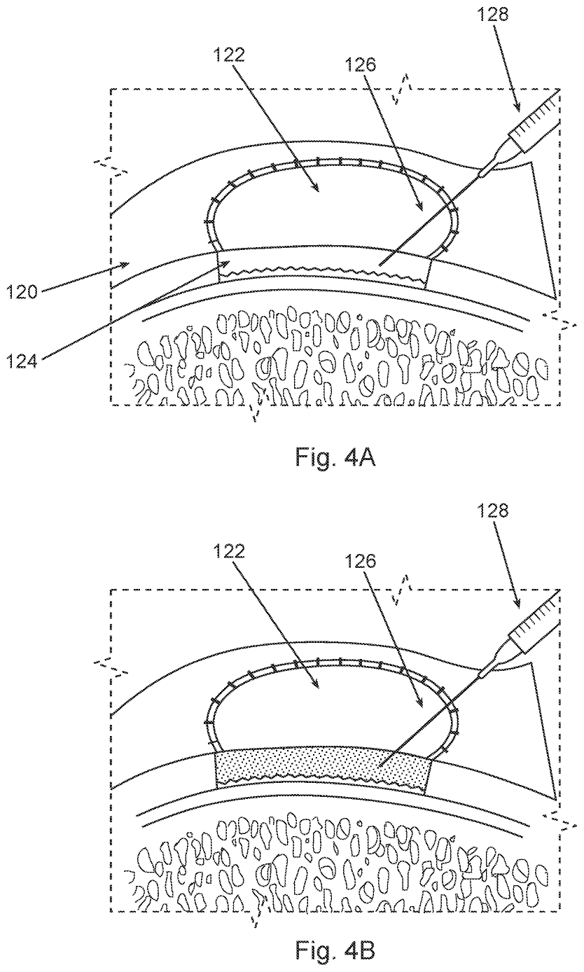

Referring now to FIG. 4A and FIG. 4B illustrating and exemplary embodiment for the treatment of a damage in cartilage. The treatment according to this embodiment is an adaptation of a treatment method generally describe in Markis EA et al. (Nature Reviews. Rheumatology, 23 Sep. 2014, 11(1):21-34)—see, for example, FIG. 1 of this reference. Generally, a cartilage 120 with a full-thickness focal chondral lesion 122 is debrided to ensure healthy, stable margins and after some further treatment the lesion is covered is covered with a periosteal flap or collagen membrane 124. A tube 126 is inserted between the membrane and the lesion and coagulating whole blood withdrawn from the patient, delivered from applicator 128, which is this specific case is a syringe, is introduced into the gap 130 between the membrane and the lesion while the tube is pulled out to create a coagulated blood mass 132 in this site, that may aid in the healing process.

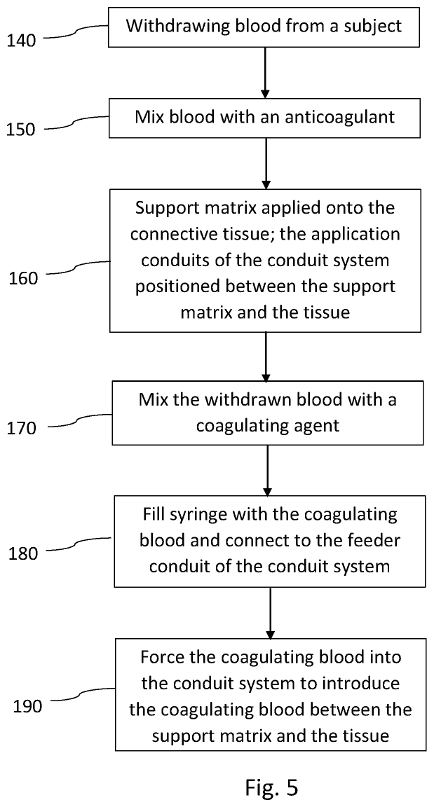

Referring now to FIG. 5, which is a block diagram of procedure according to an exemplary embodiment of this disclosure, at first step 140, whole blood is withdrawn from a subject, typically using standard blood withdrawal techniques from venous blood, e.g., by the use of a vacutainer where the withdrawn blood is collected. At step 150 the blood is mixed with an anticoagulant, that may be a priori included in the blood collection container, e.g., the vacutainer, as generally practiced in the art.

At step 160, support matrix, which may, for example, be made of collagen is applied on a damaged portion of connective tissue, with the applicator tubes at the distal portion of the conduit system, for example that shown in FIG. 3, being positioned in between the support matrix and the tissue.

At step 170, withdrawn blood is mixed with a coagulating agent, being one or more coagulating agents or anti-anticoagulants, e.g., those mentioned above, to initiate coagulation of the whole blood, and then, at 180, a syringe is filled with this coagulating blood, the syringe being then connected to the feeder conduit. In some embodiments the coagulation agent is comprised in the syringe and steps 170 and 180 are, consequently, essentially combined. Subsequently, at 190, the syringe is actuated to force the coagulating blood into the conduit system to introduce the coagulating blood between the support matrix and the tissue. By gradually withdrawing the conduit system during such introduction, the coagulating blood is distributed over the damaged portion and may be deposited at a substantially uniform manner.

The order of steps may change. For example, by some embodiments step 160 may be after step 180.

1. A kit for use in treating a damaged tissue portion of a subject, the kit comprising:
one or more blood withdrawal devices for withdrawing blood from the subject;
one or more blood collection receptacles for receiving the blood withdrawn from the subject;
a device for mixing of the withdrawn blood with one or more coagulation agents to initiate coagulation of the withdrawn blood;
a support matrix made of or comprises collagen or a degradable polymeric material, wherein the support matrix is a patch mesh, sheet, membrane, or film for placing onto the damaged tissue portion; and
one or more conduits couplable to a blood applicator at their proximal end and having one or more openings at a distal end for delivering coagulating blood from the applicator to a site between a support matrix and said portion.

2. The kit of claim 1, comprising one or more applicators couplable to the one or more conduits for delivering coagulating blood into said conduits.

3. The kit of claim 2, comprising a support matrix.

4. The kit of claim 3, wherein the support matrix is a patch mesh, membrane, film, tube or wrap.

5. The kit of claim 4, wherein said support matrix is made of collagen.

6. The kit of claim 5, wherein the one or more conduits are conduit system that comprises a feeding conduit that branches into two or more downstream application conduits for delivering the blood to said site.

7. (canceled)

8. The kit of claim 1, for use in a method for treating, inducing growth, or regenerating a damaged connective tissue portion, the method comprising:
(i) fitting a support matrix onto a damaged region of the connective tissue;
(ii) placing a distal end of one or more conduits between the damaged region of the connective tissue and the support matrix,
(iii) withdrawing whole blood from the subject;
(iv) mixing the subject's blood with one or more coagulation agents;
(v) prior to complete coagulation of the blood, injecting the blood with the coagulation agent through a proximal end of the one or more conduits;
and
(vi) permitting the blood to coagulate between the damaged connective tissue and the support matrix.

9. The kit of claim 8 wherein steps (iii) and (iv) are performed prior to or concomitantly with step (i) and (ii).

10. (canceled)

11. (canceled)

12. (canceled)

13. (canceled)

14. (canceled)

15. (canceled)

16. (canceled)

17. (canceled)

18. (canceled)

19. A method for treating a damaged connective tissue portion in a subject or inducing growth of connective tissue fibers across a damaged connective tissue portion in a subject in need thereof, comprising:
(a) fitting a support matrix onto said portion;
(b) placing a distal end of one or more conduits between said portion and the support matrix; and
(c) injecting a coagulating blood mass through a proximal end of the one or more conduits in between said portion and said support matrix, such that it accumulates and subsequently coagulates between the damaged portion of the connective tissue and the support matrix.

20. The method of claim 19, wherein the one or more conduits are retraced during injection.

21. The method of claim 19, wherein said coagulating blood mass is a mixture of whole blood withdrawn from the subject and one or more coagulating agents, and wherein said introducing is performed prior to complete coagulation of the mixture.

22. The method of claim 19, wherein said connective tissue is a tendon a ligament or a cartilage.

23. The method of claim 19, wherein said support matrix is a patch mesh, sheet, membrane, film, tube or wrap.

24. The method of claim 19, wherein the one or more conduits are connectable to an applicator at its proximal end and having one or more openings at a distal end for introducing the of coagulating blood in between the damaged portion of the connective tissue and the support matrix.

25. The method of claim 19, wherein the one or more conduits comprises a feeding conduit that branches into two or more downstream application conduits for delivering the blood in between the damaged portion of the connective tissue and the support matrix.
